(19) United States
(12) Patent Application Publication
(10) Pub. No.: US 2013/0102999 A1
(43) Pub. Date: Apr. 25, 2013

(54) PLEURODESIS CATHETER WITH COATED AND SHIELDED TIP

(71) Applicants: Anthony M. Looper, Lake Zurich, IL (US); Kelly Landsman, Milwaukee, WI (US); John A. Krueger, Muskego, WI (US)

(72) Inventors: Anthony M. Looper, Lake Zurich, IL (US); Kelly Landsman, Milwaukee, WI (US); John A. Krueger, Muskego, WI (US)

(21) Appl. No.: 13/280,865

(22) Filed: Oct. 25, 2011

(51) Int. Cl. A61L 29/16; B05D 1/32; B05D 7/00; A61M 25/01; B05D 5/00

(52) U.S. Cl. 604/528; 604/523; 427/2.3

(57) ABSTRACT

System and components for inducing pleurodesis while minimizing likelihood of tissue damage from direct application of pleurodesis-enhancing materials. A tube device is provided that is configured to elute pleurodesis-enhancing material. The device includes one or more features configured to prevent or lessen the likelihood that the pleurodesis-enhancing material comes into direct and concentrated contact with patient tissue when a distal length of the device is disposed indwelling a patient.

### TECHNICAL FIELD

Embodiments of the present invention relate to the field of removing peritoneal ascites, pleural effusion fluids, and the like. More particularly, embodiments of the present invention relate to a catheter device that can perform a drainage function and a sclerosis-inducting function while protecting tissue from direct exposure to concentrated sclerotic agents.

### BACKGROUND

Ascites describes an accumulation of fluid in the peritoneal cavity. Pleural effusion refers to the effusion of fluid into the pleural space. Both excess fluid accumulation conditions may be treated with a drainage apparatus of the type shown in FIG. 1. The apparatus 100 is shown as installed in a patient body and includes a drainage container 114. The drainage container 114 is removably attached by a proximal tube 110 at a valve 60 to a distal catheter 12. The valve 60 may be configured in any number of ways known in the art for attaching catheters together in a fluid-patent manner, (which may include a two-part valve), and the proximal portion attached to the distal catheter 12 may be configured to be self-sealing when disconnected from the proximal tube 110. The proximal end portion of the distal catheter 12 is shown indwelling the patient, disposed through the body wall 21 into an intra-body space 23, which may be—for example—a pleural, peritoneal, or other body lumen. That proximal portion includes a sealing cuff 19 and a flexible fluid-intake length 14 including apertures 18, shown in the intra-body space 23. This structure may be better understood with reference to U.S. Pat. No. 5,484,401, which is incorporated herein by reference, and with reference to commercial products marketed under the name PleurX by CareFusion® of San Diego, Calif.

The pleural space normally contains approximately 5 to 20 ml of fluid. The pH, glucose and electrolytes of the fluid are equilibrated with plasma, but the fluid is relatively protein-free. The fluid is the result of the hydrostatic-oncotic pressure of the capillaries of the parietal pleura. About 80-90% of the fluid is reabsorbed by the pulmonary venous capillaries of the visceral pleura, and the remaining 10-20% is reabsorbed by the pleural lymphatic system. The turnover of fluid in the pleural space is normally quite rapid-roughly 35 to 75% per hour, so that 5 to 10 liters of fluid move through the pleural space each day.

A disruption in the balance between the movement of fluid into the pleural space and the movement of fluid out of the pleural space may produce excessive fluid accumulation in the pleural space. Such disruptions may include, for example, (1) increased capillary permeability resulting from inflammatory processes such as pneumonia, (2) increased hydrostatic pressure as in congestive heart failure, (3) increased negative intrapleural pressure as seen in atelectasis (partial or total lung collapse), (4) decreased oncotic pressure as occurs in the nephrotic syndrome with hypoalbuminemia, and (5) increased oncotic pressure of pleural fluid as occurs in the inflammation of pleural tumor growth or infection. Pleural effusion is particularly common in patients with disseminated breast cancer, lung cancer or lymphatic cancer and patients with congestive heart failure, but also occurs in patients with nearly all other forms of malignancy.

The clinical manifestations of pleural effusion include dyspnea, cough and chest pain which diminish the patient's quality of life. Although pleural effusion typically occurs toward the end of terminal malignancies such as breast cancer, it occurs earlier in other diseases. Therefore relieving the clinical manifestations of pleural effusion is of a real and extended advantage to the patient. For example, non-breast cancer patients with pleural effusion have been known to survive for years.

There are a number of treatments for pleural effusion. If the patient is asymptomatic and the effusion is known to be malignant or paramalignant, treatment may not be required. Such patients may develop progressive pleural effusions that eventually do produce symptoms requiring treatment, but some will reach a stage where the effusions and reabsorption reach an equilibrium that is still asymptomatic and does not necessitate treatment.

Pleurectomy and pleural abrasion is generally effective in obliterating the pleural space and, thus, controlling the malignant pleural effusion. This procedure is done in many patients who undergo thoracotomy for an undiagnosed pleural effusion and are found to have malignancy, since this would prevent the subsequent development of a symptomatic pleural effusion. However, pleurectomy is a major surgical procedure associated with substantial morbidity and some mortality. Therefore, this procedure is usually reserved for patients with an expected survival of at least several months, who are in relative good condition, who have a trapped lung, or who have failed a sclerosing agent procedure.

In general, systemic chemotherapy is disappointing for the control of malignant pleural effusions. However, patients with lymphoma, breast cancer, or small cell carcinoma of the lung may obtain an excellent response to chemotherapy. Another approach to removing fluid from the pleural space has been to surgically implant a chest tube. Such tubes are commonly quite rigid and fairly large in diameter and are implanted by making a surgical incision and spreading apart adjacent ribs to fit the tube into place. Such procedures are painful to the patient, both initially when the chest tube is inserted and during the time it remains within the pleural space.

Thoracentesis is a common approach to removing pleural fluid, in which a needled catheter is introduced into the pleural space through an incision in the chest cavity and fluid is positively drawn out through the catheter using a syringe or a vacuum source. The procedure may also include aspiration utilizing a separate syringe. There are a number of difficulties in thoracentesis, including the risk of puncturing a lung with the catheter tip or with the needle used to introduce the catheter, the risk of collapsing a lung by relieving the negative pressure in the pleural space, the possibility of aggravating the pleural effusion by stimulating fluid production in the introduction of the catheter, and the risk of infection. One of the primary difficulties with ordinary thoracentesis procedures is that fluid reaccumulates in the pleural space relatively quickly after the procedure is performed, and so it is necessary to perform the procedure repeatedly—as often as every few days.

Modern pleural and peritoneal drainage systems have made it possible for patients to use devices like those illustrated in FIG. 1 to conduct drainage on periodic office or hospital visits. For patients who experience recurrent effusions, repeat drainage procedures at a clinical facility can be avoided by the installation of an indwelling tunneled catheter that can be drained at home. In addition, for some patients it may be desirable to administer a substance or provide a therapeutic intervention to the area where the catheter is inserted. For example, in patients with pleural effusion who have a lung that re-expands upon drainage, fusion of the visceral and parietal pleura is a treatment option that eliminates at least a portion of the pleural cavity and thus eliminates the space where the fluid accumulates. This procedure is called pleurodesis and can be accomplished through inciting the patient foreign body response and draining the effusion. Mechanical or chemical means can be used to cause the irritation. In other instances, continuous delivery of medication or cell signaling molecules may be desired in the area where the catheter resides.

Chemical pleurodesis may use irritants and/or antibiotic materials (also known as sclerotic agents or accelerodesis agents) that may also provide mechanical irritation to trigger cell growth and/or resist infection. Examples of materials known and used include bleomycin, tetracycline, and povidone iodine. As another example, a slurry of talc can be introduced into the pleural space. These materials generally are introduced through a thoracic drainage catheter. The instilled chemicals cause irritation between the parietal and the visceral layers of the pleura which closes off the space between them and prevents further fluid from accumulating. Chemical pleurodesis may be a painful procedure, so patients are often premedicated with a sedative and analgesics. A local anesthetic may be instilled into the pleural space, or an epidural catheter may be placed for anesthesia. Generally, to be effective, introduction of structures and materials for pleurodesis desirable will create irritation and then keep the space dry. In order to establish pleurodesis, it is preferable that the parietal and visceral layers of the pleura remain in juxtaposition. As such, it is preferable that when mechanical and/or chemical irritation is complete a drainage tube will remain in place to remove the fluid over the time it takes for the adhesion accomplishing pleurodesis to occur.

During chemical pleurodesis using a catheter surface-coated with (or otherwise eluting) a pleurodesis-inducing sclerotic agent, it may be preferable to minimize or prevent direct and/or concentrated contact of the agent with patient tissue. Accordingly, it would be advantageous to provide a catheter surface-coated with (or otherwise eluting) a pleurodesis-inducing sclerotic agent that is configured to minimize or prevent direct and/or concentrated contact of the agent with patient tissue.

### BRIEF SUMMARY

In one aspect, embodiments may include embodiments of catheter devices configured to develop a sclerotic agent from a coated surface while minimizing direct tissue contact with a sclerotic agent coating. Different embodiments may include one or more of mechanical barriers or other structures configured to impede tissue from direct contact with a catheter surface, reliance upon fluid diffusion from a protected sclerotic agent coating, and/or other structures or instrumentalities.

### BRIEF DESCRIPTION OF THE DRAWINGS

FIG. 1 illustrates a drainage apparatus as known in the prior art;

FIG. 2 shows one embodiment of a distal indwelling portion of a pleurodesis-promoting catheter;

FIG. 3 shows another embodiment of a distal indwelling portion of a pleurodesis-promoting catheter;

FIGS. 4-4C show other embodiments of a distal indwelling portion of a pleurodesis-promoting catheter;

FIG. 5 shows another embodiment of a distal indwelling portion of a pleurodesis-promoting catheter;

FIG. 6 shows another embodiment of a distal indwelling portion of a pleurodesis-promoting catheter; and

FIG. 7 shows another embodiment of a distal indwelling portion of a pleurodesis-promoting catheter.

### DETAILED DESCRIPTION

Embodiments generally are described with reference to the drawings in which like elements are generally referred to by like numerals. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments of the present invention, such as—for example—conventional fabrication and assembly.

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly indicates otherwise. Throughout the specification, the terms “distal” and “distally” shall denote a position, direction, or orientation that is generally away from the physician and/or toward the patient. Accordingly, the terms “proximal” and “proximally” shall denote a position, direction, or orientation that is generally towards the physician and/or away from the patient.

Within a device for accelerating and/or enhancing pleurodesis, it may be desirable to provide a distal indwelling portion of the tube that is coated at least partially with a substance that is intended to be delivered to the body over an extended period of time in a diluted, consistent, and/or titrated manner. One example of such a system maybe a tube body configured for pleurodesis of the pleural space by means of a sclerosing agent such as, for example, silver nitrate. In these instances, it is preferable that the silver nitrate coating in its base/concentrated form not contact the surrounding tissue directly due to its high concentration and potential tissue reactions thereto. The coating most preferably will be eluted or otherwise be released over time from the catheter. Other suitable sclerotic agents may include antimicrobial agents, or other materials configured for inducing pleurodesis (e.g., polyvinylpyrrolidone (PVP), talc (e.g., as a slurry), bleomycin, mitoxantrone, mitomycin, thiotpea, cytarabine, quinacrine, tetracycline (defined herein to include tetracycline derivative such as doxycycline and minocycline), OK432 (Streptococcus pyogenes type A3), SSAg (Staphylococcus aureus superantigen), fibrin glue, povidone iodine (PVP-I), autologous “blood patch,” or any combination thereof).

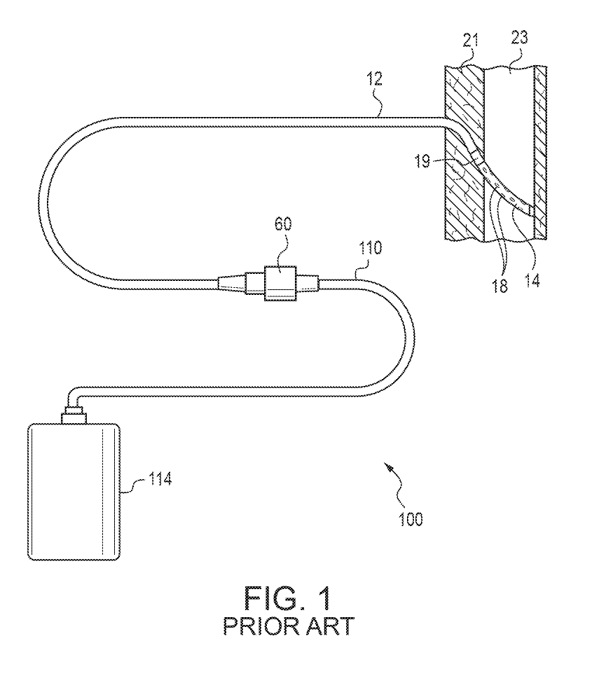

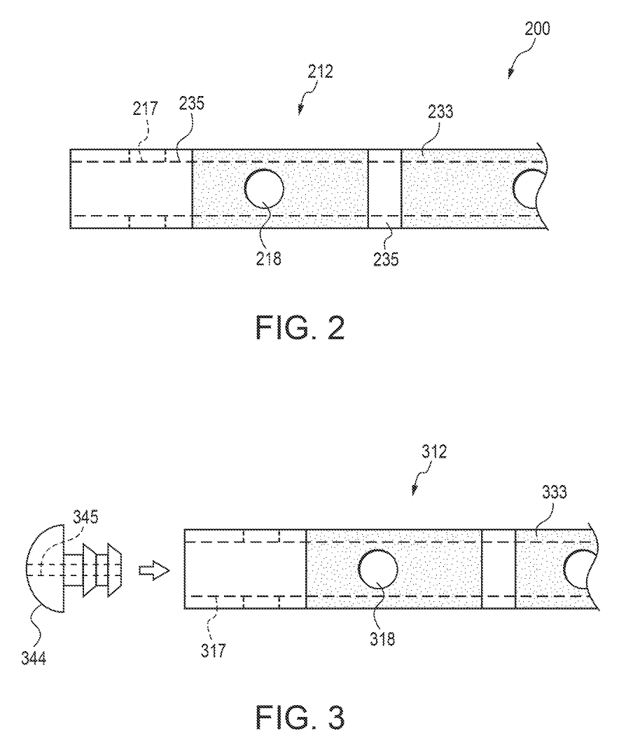

FIG. 2 shows one embodiment of the distal indwelling portion 202 of a drainage catheter device 200. A system including a drainage catheter 200 may generally be configured in the manner shown in FIG. 1, but the embodiment of FIG. 2 and following in FIGS. 3-7 are focused upon the construction of a distal length of the device 200. A proximal end portion of the illustrated distal catheter length 212 preferably is configured for indwelling a pleural or other intra-body space inside a patient. At least one aperture 218 configured to facilitate drainage from the space is provided through a wall of the distal catheter length 212.

As described above, pleurodesis may be induced or enhanced by providing a sclerotic agent. One means for this is as a coating on at least one internal and/or external surface to the distal catheter length 212 configured to indwell the patient. However, it may be preferable that such a coating not directly contact patient tissue, as the concentration of a sclerotic agent coating may have an excessively irritating or other adverse effect. Instead, it is preferable that the sclerotic agent of the coating be allowed to diffuse through the pleural or other space and thereby to contact the patient tissue in a less concentrated manner. One means for providing this is described with reference to FIG. 2, and will readily be understood by those of skill in the art.

FIG. 2 shows a distal catheter length 212 of an elongate flexible tube. The distal length includes at least one drainage aperture 218, embodied here as a plurality of apertures. The apertures 218 are open in fluid communication with a central body lumen 217 of the catheter length 212. A lengthwise portion of the distal length 212 is configured as an eluting portion 233 coated with a sclerotic agent that will elute into a body space. During construction of the distal portion, selected surfaces 235 of the eluting portion may be masked to prevent those surfaces from being coated. In FIG. 2, the masked, uncoated portion 235 includes a distal end length immediately adjacent the distal terminus of the tube.

The regions to be masked and thereby left uncoated are most preferably selected and configured to be regions of the distal length most likely to directly contact patient tissue when installed into a body space. For different body spaces, patient anatomy, and/or device sizes, the masked regions may be custom-designed, or may be configured generally for a desired application. A distal terminus region or any other region or plurality of regions of the eluting may be masked and thereby remain uncoated with sclerotic agent. Regions that are most desired to be masked and remain uncoated may be identified by a treating physician, by observation of tube position in a patient with a prior uncoated tube, or by other means.

In this or other embodiments, one or more surfaces of the tube lumen 217 may be provided with a coating or otherwise elutable application of a sclerotic agent. This location will prevent or decrease the likelihood of direct and/or concentrated contact between the sclerotic agent and patient tissue when the device is disposed indwelling a patient. The sclerotic agent eluent can travel through the drainage apertures 218 and/or a distal end opening to promote pleurodesis.

FIG. 3 shows a distal portion of another drainage catheter device embodiment including a distal catheter length 312 of an elongate flexible tube. The distal length includes at least one drainage aperture 318 and a distal end opening of the tube lumen 317 configured to engagingly receive a weighted element configured as a plug 344. The plug 344 may be constructed of metal material, ceramic material, or a combination thereof. In other embodiments, the weighted element may be configured as a ring or other shape attached to and weighting the distal catheter length 312. The plug may include a plug lumen 345 extending longitudinally therethrough to provide a path of fluid communication with the tube lumen 317.

This weighted embodiment most preferably is configured such that the weight 344 will generally orient a coated eluting portion 333, which is coated with a sclerotic agent, away from direct contact with patient tissue when the distal catheter length 312 is installed in a patient. For example, when the distal catheter length 312 is installed as indwelling in a lateral or dorsal pleural space of a patient, the eluting portion 333 (embodied as a coated distal region) will generally be held away from direct contact with patient tissue so that the sclerotic agent contacts that tissue to promote pleurodesis only in an at least somewhat diluted, diffused form rather than permitting direct contact with the surface coating of the eluting portion 333. Even if some direct contact is permitted, it is strongly preferred that the surface coating of the eluting portion 333 not directly contact major blood vessels of the mediastinum. Those of skill will appreciate in view of the present disclosure that appropriate placement of this embodiment with its weighted distal end will facilitate this orientation configured to minimize likelihood of direct contact.

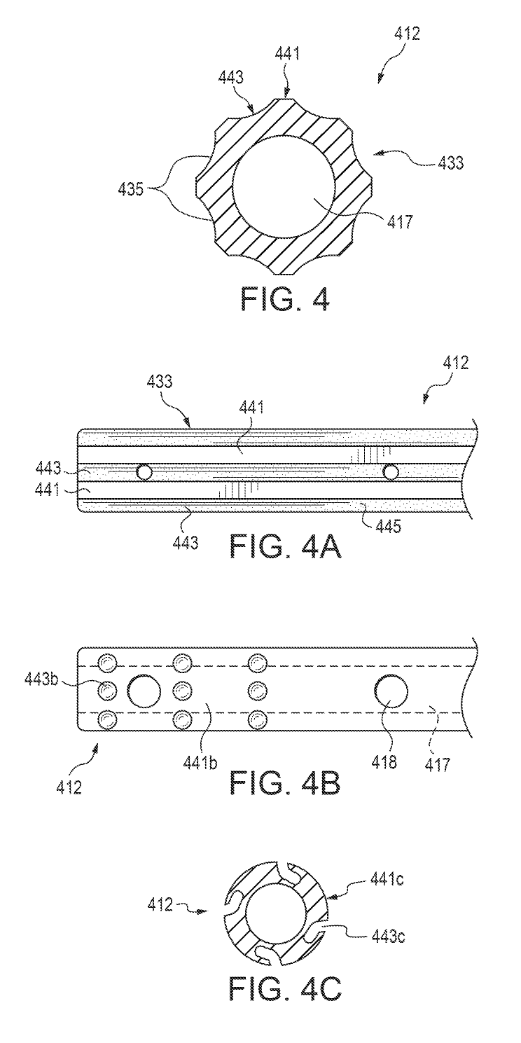

FIG. 4 shows a transverse cross-sectional view of a catheter device embodiment. Specifically, FIG. 4 shows a section view of an eluting portion 433 of a distal catheter length 412 including a lumen 417. The outer surface includes at least one relatively raised external surface 441, and at least one relatively depressed external surface 443 adjacent the at least one relatively raised external surface 441. A sclerotic agent coating 435 is disposed upon the at least one relatively depressed external surface 443 at a greater concentration than upon the at least one relatively raised external surface 441, which may not include any sclerotic agent coating.

In this embodiment, the relatively raised and depressed surfaces are formed as channels 443 and dividers 441. FIG. 4A shows a side view of the distal catheter length 412, where the channels are shown as substantially longitudinally straight. However, in other embodiments, relatively raised and depressed surfaces embodied as channels may wind helically along a surface (exterior or interior) of the catheter length 412. FIG. 4B shows a side view of the distal catheter length 412, which has the same cross-sectional profile shown in FIG. 4, but where the relatively depressed surfaces are configured as dimples 443b in the surface separated by relatively raised surface 441b, but which are distinct from the drainage apertures 418 that are in fluid communication with longitudinal central lumen 417. In another embodiment, shown in the transverse section of FIG. 4C, the depressed surfaces are configured as cavities (each at least one depressed surface comprising at least one cavity) 443c that are coated with sclerotic agent (not shown) and separated by relatively raised surfaces 441c. It should be appreciated that these configurations will decrease the likelihood of direct contact between adjacent tissue and the sclerotic agent when the distal catheter length 412 is disposed as indwelling in a patient's body space. In each of these embodiments, when the distal catheter length 412 is disposed as indwelling in a patient's body space, it will generally be preferred to provide a concentration of the sclerotic agent in a suprasurface region immediately adjacent the at least one relatively depressed surface is greater than a concentration of the sclerotic agent in a suprasurface region immediately adjacent the at least one relatively raised surface. That is, to the extent that a suprasurface region immediately above the catheter length's surface is capable of carrying sclerotic agent eluted from the device, the concentration immediately adjacent/above the relatively depressed region(s) will be greater than the concentrations immediately adjacent/above the relatively raised region(s).

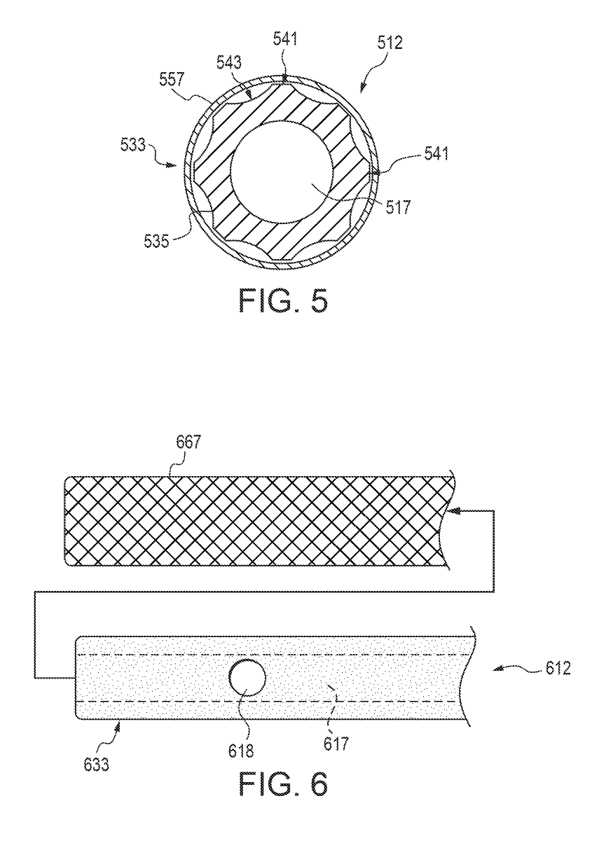

FIG. 5 shows another varied embodiment of the device of FIGS. 4-4C. FIG. 5 shows a transverse cross-sectional view of a catheter device embodiment. Specifically, FIG. 5 shows a section view of an eluting portion 533 of a distal catheter length 512 including a lumen 517. The outer surface includes at least one relatively raised external surface 541, and at least one relatively depressed external surface 543 adjacent the at least one relatively raised external surface 541. A sclerotic agent coating 535 is disposed upon at least one relatively depressed external surface 543 at a greater concentration than upon the at least one relatively raised external surface 541, which may not include any sclerotic agent coating. The outer circumference of the eluting portion 533 is encircled by a thin tube 557. The thin tube 557 may be perforated, porous, permeable to the sclerotic agent, or any combination thereof. Those of skill in the art will appreciate that various polymeric and other materials well-known in the art may be used to construct such a tube. As with the other embodiments described herein, the thin tube 557 will preferably prevent direct and/or otherwise concentrated contact of sclerotic agent coating with tissue around the eluting portion 533 when it is indwelling a patient. However, the tube 557 will permit a low concentration of the sclerotic agent to pass therethrough to promote pleurodesis.

FIG. 6 shows another embodiment of a catheter device configured to enhance pleurodesis while minimizing direct tissue contact with a sclerotic agent on a surface of the catheter device. FIG. 6 shows a side view of a catheter device embodiment including an outer sheath. The sheath may be constructed of, for example silicone, TPU (thermoplastic polyurethane), stainless steel, nylon, implantable foam, polypropylene, urethane, PVC, or other biocompatible material that may be constructed, for example, as a porous or fenestrated tube, a stent-like cage, or other configuration (such that some manifestations of this embodiment may share common properties and structure with the embodiment described above with reference to FIG. 5. Specifically, FIG. 6 shows a side view of an eluting portion 633 of a distal catheter length 612 including a lumen 617 with which drainage apertures 618 are in fluid communication. A sclerotic agent coating (indicated by shaded surface) is disposed upon an external eluting surface 633 of the distal catheter length 612. The outer circumference of the eluting portion 633 is encompassed by a spacing tube 667 (shown in exploded view in FIG. 6 for clarity of illustration).

The tube 667 may be configured as mesh, woven, braided, or the like (including attached but non-interlaced structures) and is porous such that it is permeable to the sclerotic agent of the eluting surface 633 while forming a spacing barrier configured to prevent direct contact of adjacent tissue with the sclerotic agent of the eluting surface 633. Those of skill in the art will appreciate that various metallic, polymeric, and other materials well-known in the art may be used to construct such a tube (which may be constructed similarly to any number of stent configurations known in the art). As with the other embodiments described herein, the spacer tube 667 preferably will prevent direct and/or otherwise concentrated contact of sclerotic agent coating with tissue around the eluting portion 633 when the device is indwelling a patient. However, the tube 667 will permit a low concentration of the sclerotic agent to pass therethrough to promote pleurodesis. The spacer tube 667 may be attached along the eluting surface 633 and/or may be spaced apart from it by being constructed with an inner diameter that is larger than the outer diameter of the eluting portion 633.

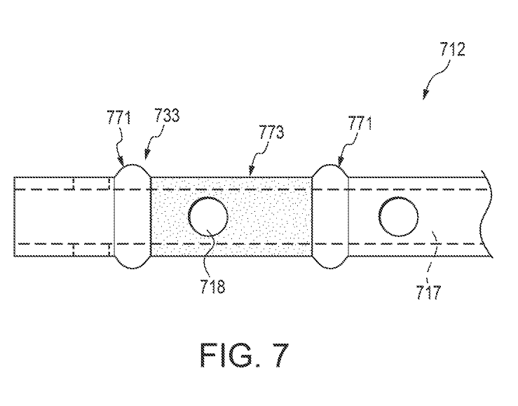

FIG. 7 shows an external side view of a catheter device embodiment. Specifically, FIG. 7 shows a section view including an eluting portion 733 of a distal catheter length 712 that includes a lumen 717. As in the other embodiments, drainage apertures 718 may be included to provide fluid communication through the catheter tube wall with the lumen 717. The outer surface includes at least two relatively raised external surfaces 771, with at least one relatively depressed external surface 773 adjacent and between the at least two relatively raised external surfaces 771. A sclerotic agent coating (indicated by shaded surface) is disposed upon the at least one relatively depressed external surface 773 at a greater concentration than upon the at least two relatively raised external surfaces 771, one or both of which may not include any sclerotic agent coating. The raised surfaces are shown here as bumps or concentric ridges, but they may be configured as individual raised protrusions (e.g., mushroom-shaped, bristle-shaped, or other shape disposed around the outer tube circumference and configured to minimize or prevent direct surface contact of a tube portion with adjacent tissue).

The at least two relatively raised external surfaces 771 are disposed at least generally circumferentially around the outer surface of the distal catheter length 712. The at least two relatively raised external surfaces 771 may be parallel (as shown in FIG. 7) or non-parallel. The at least two relatively raised external surfaces 771 may be generally perpendicular to the longitudinal central axis of the distal catheter length, or one or more of the at least two relatively raised external surfaces 771 may be angled relative to that perpendicular axis.

It should be appreciated that these configurations will decrease the likelihood of direct contact between adjacent tissue and the sclerotic agent when the distal catheter length 712 is disposed as indwelling in a patient's body space. In each of these embodiments, when the distal catheter length 712 is disposed as indwelling in a patient's body space, it will generally be preferred to provide a concentration of the sclerotic agent in a suprasurface region immediately adjacent the at least one relatively depressed surface is greater than a concentration of the sclerotic agent in a suprasurface region immediately adjacent the at least one relatively raised surface. That is, to the extent that a suprasurface region immediately above the catheter length's surface is capable of carrying sclerotic agent eluted from the device, the concentration immediately adjacent/above the relatively depressed region(s) will be greater than the concentrations immediately adjacent/above the relatively raised region(s).

The spacing between the at least two relatively raised external surfaces 771 preferably is configured relative to the height of each of the at least two relatively raised external surfaces 771 such that tissue (e.g., pleural tissue) likely to be adjacent to the distal catheter length 712 will generally be prevented from, or will at least have a reduced likelihood of, direct contact with the eluting surface 773 between the at least two relatively raised external surfaces 771. In certain embodiments, the at least two raised external surfaces 771 may be configured with a height that is will minimize direct contact between the eluting surface 773 and adjacent tissue. In some embodiments, the sclerotic agent may be distributed in a gradient fashion, with a higher concentration at or near the base of a raised surface 771 (where it may be more shielded from direct tissue contact) and lessening concentration further away from the raised surface 771.

Those of skill in the art will appreciate that embodiments not expressly illustrated herein may be practiced within the scope of the present invention, including that features described herein for different embodiments may be combined with each other and/or with currently-known or future-developed technologies while remaining within the scope of the claims presented here. For example, the various physical structures disclosed may also provide mechanical irritation promoting a desired sclerotic effect, and the structures and components disclosed herein may be combined with each other or other features. Although specific terms are employed herein, they are used in a generic and descriptive sense only and not for purposes of limitation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment of the invention.

We claim:

1. A drainage catheter device comprising:
an elongate flexible tube body including
a distal length configured to indwell a patient; and
a body lumen extending longitudinally through at least a lengthwise portion of the distal length, the lumen substantially defined by an inner diameter surface of the tube body;
the distal length including at least one aperture disposed through a wall of the body, and in fluid communication with the body lumen;
wherein at least one portion of the distal length is configured as an eluting portion that includes at least one surface constructed to elute a sclerotic agent; and
wherein at least one structure is provided and configured to decrease a probability of direct contact between the eluting portion and a surface external of the device disposed immediately adjacent the eluting portion.

2. The catheter device of claim 1, wherein the eluting portion comprises a sclerotic agent.

3. The catheter device of claim 1, eluting portion comprises a coating of silver nitrate, talc, bleomycin, tetracycline, povidone iodine, polyvinylpyrrolidone (PVP), mitoxantrone, mitomycin, thiotpea, cytarabine, quinacrine, OK432 (Streptococcus pyogenes type A3), SSAg (Staphylococcus aureus superantigen), fibrin glue, autologous “blood patch,” or any combination thereof.

4. The catheter device of claim 1, wherein at least one external surface immediately adjacent the eluting portion is configured to exclude any sclerotic agent.

5. The catheter device of claim 1, wherein at least one external surface region of the eluting portion is configured to exclude any sclerotic agent.

6. A method of making the catheter device of claim 5, the method comprising steps of:
providing the body;
masking with a masking material the at least one external surface region;
applying a coating of a sclerotic agent; and
removing the masking material.

7. The catheter of claim 1 wherein the at least one structure comprises:
a weighted element disposed near a distal end terminus of the body, wherein the weighted element is configured to orient the at least one eluting portion away from direct contact with patient tissue when the device is disposed in a pleural space.

8. The catheter device of claim 7, wherein the weighted element is configured as a plug disposed in a distal end terminus of the body lumen.

9. The catheter device of claim 8, wherein the plug is constructed of metal material, ceramic material, or a combination thereof.

10. The catheter device of claim 1, wherein the at least one eluting portion comprises:
at least one relatively raised external surface and at least one relatively depressed external surface adjacent the at least one relatively raised external surface;
wherein a sclerotic agent is disposed upon the relatively depressed external surface.

11. The catheter device of claim 10, wherein the at least one relatively raised external surface comprises at least two generally longitudinal external raised surfaces and the at least one relatively depressed surface comprises a channel between the at least two generally longitudinal external raised surfaces.

12. The catheter device of claim 10 wherein the at least one relatively depressed surface comprises one or more of at least one channel, at least one dimple, and at least one cavity.

13. The catheter device of claim 10 wherein a concentration of the sclerotic agent in a suprasurface region immediately adjacent the at least one relatively depressed surface is greater than a concentration of the sclerotic agent in a suprasurface region immediately adjacent the at least one relatively raised surface.

14. The catheter device of claim 10, further comprising a thin tube disposed around at least a part of the at least one eluting portion, wherein the tube is configured to allow passage therethrough of the sclerotic agent.

15. The catheter device of claim 14, wherein the tube is configured as perforated, permeable to the sclerotic agent, or a combination thereof.

16. The catheter device of claim 1, further comprising a permeable sheath disposed around and substantially covering the at least one eluting portion, wherein the sheath is configured to allow passage therethrough of a sclerotic agent disposed on the at least one eluting portion.

17. The catheter device of claim 16, wherein the permeable sheath comprises at least one of a mesh material, a perforated material, or a combination thereof.

18. The catheter device of claim 1, wherein the eluting portion further comprises at least two circumferential raised surfaces each having a greater outer diameter than an immediately adjacent portion of the eluting portion, and wherein a sclerotic agent is disposed in a higher concentration in a region between the at least two circumferential raised surfaces than upon an outer surface of the at least two circumferential raised surfaces

19. The catheter device of claim 18, wherein the at least two circumferential raised surfaces are substantially parallel with each other.

20. The catheter device of claim 18, wherein the at least two circumferential raised surfaces are configured with a height relative to the distance between the at least two circumferential raised surfaces that will minimize direct tissue contact with the region between the at least two circumferential raised surfaces.

21. The catheter device of claim 1, wherein the at least one eluting portion comprises the inner diameter surface, said surface at least partially coated with a sclerotic agent and configured to elute the sclerotic agent through the at least one aperture.

22. The catheter device of claim 1, wherein the at least one aperture comprises a plurality of apertures.
